(19) United States
(12) Patent Application Publication
(10) Pub. No.: US 2021/0020311 A1
(43) Pub. Date: Jan. 21, 2021

(54) Method for Enhanced Medical Data Pattern Recognition

(71) Applicant: Richard G. Johnson, Pittsburgh, PA (US)

(72) Inventor: Richard G. Johnson, Pittsburgh, PA (US)

(21) Appl. No.: 16/930,982

(22) Filed: Jul. 16, 2020

Related U.S. Application Data
(60) Provisional application No. 62/875,259, filed on Jul. 17, 2019.

(51) Int. Cl. G16H 50/20; A61B 5/0205; A61B 5/00; G16H 50/30

(52) U.S. Cl. CPC G16H 50/20; A61B 5/0205; A61B 5/4857; A61B 5/14551; A61B 5/7415; G16H 50/30; A61B 5/02055; A61B 5/743

(57) ABSTRACT

The present invention provides a method for enhanced medical diagnostic data pattern recognition in an individual or in a population of individuals. The method comprises compiling a dataset consisting of at least seven essential vital signs of an individual or population of individuals, wherein the dataset is compiled and mapped in at least three ways: (1) as a mathematical tensor; (2) as a visual graph; or (3) as a musical composition. Essential vital signs can include systolic pressure values of 60-140; diastolic pressure values of 40-85; time of day values of 1-24; oximeter data values of 92-99; date data values translated to adjusted circadian process; pulse data values of 40-90; and weight values.

### CROSS REFERENCE TO RELATED APPLICATIONS

This application claims priority to U.S. Provisional Patent Application No. 62/875,259, filed Jul. 17, 2019, which is incorporated herein by reference in its entirety.

### FIELD OF THE INVENTION

The present invention relates to medical diagnostics and, in particular, to enhanced medical data pattern recognition diagnostics.

### BACKGROUND OF THE INVENTION

Medical diagnostics is the process of determining which disease or condition explains an individual's symptoms and signs. The information required for diagnosis typically is collected from a history and physical examination of an individual seeking medical care. Often, one or more diagnostic procedures, such as medical tests, are also done during the process.

Medical diagnosis often is challenging because many signs and symptoms are nonspecific. For example, redness of the skin, by itself, is a sign of many disorders, and thus does not tell the healthcare professional what is wrong. Thus, differential diagnosis, in which several possible explanations are compared and contrasted, must be performed. This involves the correlation of various pieces of information followed by the recognition and differentiation of patterns. Occasionally, the process is made easy by a sign or symptom (or a group of several) that is pathognomonic.

General components which are present in a diagnostic procedure in most of the various available methods include complementing the already given information with further data gathering, which may include questions of medical history, physical examination and various diagnostic tests.

There are a number of methods or techniques that can be used in a diagnostic procedure, including performing a differential diagnosis or following medical algorithms. In reality, a diagnostic procedure may involve components of multiple methods. For example, in pattern recognition methods, the health care provider uses experience to recognize a pattern of clinical characteristics. It mainly is based on certain symptoms or signs being associated with certain diseases or conditions, not necessarily involving the more cognitive processing involved in a differential diagnosis. This may be the primary method used in cases where diseases are obvious, or the provider's experience may enable recognition of the condition quickly. Theoretically, a certain pattern of signs or symptoms can be directly associated with a certain therapy, even without a definite decision regarding what is the actual disease, but such a compromise carries a substantial risk of missing a diagnosis which actually has a different therapy so it may be limited to cases where no diagnosis can be made.

### SUMMARY OF THE INVENTION

The present invention provides a unique method for enhanced medical diagnostic data pattern recognition of multiple data sets in an individual over a specific time period, and identification of patterns in a population of individuals by assessing multiple data sets of cohorts in the populations over a specific time period.

The method comprises the steps of (1) electing at least seven vital signs or indicators of an animal or human for which diagnosis is indicated; (2) monitoring the vital signs or indicators over time with data capture; and (3) compiling and mapping the data, using a computer, to map the data capture in a form selected from the group consisting of a mathematical tensor, a visual graph and a musical composition.

The method further comprises mapping the data capture in a form comprised of (1) a linear graph of x, y, z, as x=t\hat{ }y=P/p; x=t\hat{ }y=pBpm\hat{ }z=dP; (2) a hyperbolic graph; (3) a symbolic logic graph; or (4) a logarithmic graph.

Data capture compiling and mapping may be calculated according to the equation: x=Σ 7+y (v_{n1, n2, n3, n4, n5, n6, n7 . . .} ), wherein x is the output to a user; y=0-100; and n=individual vital sign or indicator data or time, which includes, without limitation, systolic pressure, diastolic pressure, body temperature, time elapse, oximeter, pulse, body mass, antibody titer, white cell count, T cell count, red cell count, a cancer marker, hematocrit, hemoglobin, platelet count, platelet volume, biomarkers such as immune markers or metabolic markers, and an indicator.

Data capture compiling and mapping also may be calculated according to homologic equations: If l is a face of a simplex k [symbol] K, then l [symbol] K.

For k, l∈K, k∩l [symbol] k [symbol] k∩l [symbol] l.

The at least seven essential vital signs of an individual may include, without limitation: (1) systolic pressure values of 60-140; (2) diastolic pressure values of 40-85; (3) time of day values of 1-24; (4) oximeter data values of 92-99; (5) date data values translated to adjusted circadian process; (6) pulse data values of 40-90; and (7) weight values.

### DETAILED DESCRIPTION OF THE INVENTION

The present invention provides a method comprising compiling a dataset consisting of at least seven essential vital signs of an individual, wherein the individual dataset is compiled and mapped in at least three ways: (1) as a mathematical tensor; (2) as a visual graph; or (3) as a musical composition.

Such a dataset, being a unique array, constitutes a description of an individual, and being so personalized, the dataset is a unique tensor equivalent to an individual fingerprint, and is infinitely more unique than genetic analysis of the individual.

The present invention therefore takes medical vital signs or indicators and renders them in a compiled form, for instant or prompt viewing or auditory review. The ways a computer can compile such data capture are endless—the invention is both in knowing to render such data capture in this new way, and in programming the computer to do it, such that the output to a user is reviewable by sight or sound and patterns therein are recognizable as having meaning.

With respect to compiling and mapping an individual's dataset as a mathematical tensor, it can be represented as a straightforward Einsteinian Tensorial Array, i.e., a multi-dimensional array of numbers.

With respect to compiling and mapping an individual's dataset as a visual graph, it can be either: (1) a linear graph, i.e., x, y, or x, y, z, e.g., x=t\hat{ }y=P/p; x=t\hat{ }y=pBpm\hat{ }z=dP; (2) a hyperbolic graph; (3) a symbolic logic graph; or (4) a logarithmic graph.

With respect to compiling and mapping an individual's dataset as a musical composition, each parameter can be mapped to a set-inclusive range which defines, for example and without limitation:

1. Systolic pressure values of 60-140, which can be mapped 1-9, e.g., melody notes Middle C to c′;

2. Diastolic pressure values of 40-85, which can be mapped 1-12, e.g., chromatic melody notes;

3. Time of day values of 1-24, which can be mapped to two octaves of bass notes;

4. Oximeter data values of 92-99, which can be mapped to 1-8 continuo definition;

5. Date data values translated to adjusted circadian process, which can be mapped to 2/4, ¾, 4/4, 6/8, ⅞, etc., rhythm patterns;

6. Pulse data values of 40-90, which can be mapped to 1-6 pianissimo to pffForte; and

7. Weight values, being hard to collect, can be mapped to Key Signatures.

The gravamen of the present invention is the mathematical conversion of patient data into a “gestalt,” in a way that no diagnostic reporting has ever done before. The gestalt, or “big picture,” is typically a kind of “heads up display,” in which a physician or other health care provider can see complicated data reduced to any or all of a mathematical tensor, a visual graph or even a musical composition that can be heard, instead of viewed. The importance of this approach is, in part, that individual vital signs or indicators have additive significance, beyond just “being within or out of normal range” themselves, particularly when change in sign or indicator are considered together and over time.

In other words, it is good to know whether two (or more) signs or indicators are within or outside of their normal ranges—but how does the diagnostician readily discern when both those signs or indicators, together, are significant in their variations, both in concert and as they change over time?

Those skilled in the art will immediately appreciate the utility of the described and claimed invention. An early iteration of a parallel idea, not suggestive of the present invention, was also immediately apparent to those skilled in the art—U.S. Pat. No. 6,063,586 to Jennifer Grandis, entitled “Diagnostic Protocol.” In this patent, static graphing of data in two dimensions only, of EGFR and TGFα levels, proved diagnostically significant of specified patient outcomes when data clustered in a particular quadrant of the two-dimensional graph (see, for example, FIG. 4). Nothing in U.S. Pat. No. 6,063,586 suggests, however, anything like the present invention, which multiplies the number of signs or indicators to at least seven, typically and ideally includes time as a variable, and importantly renders the compiled data as a visual or auditory output reducing multiple dimensions to a display that can be comprehended all at once.

The mathematics of compiling data sets according to the present invention is within the skill of the art, but has to the inventor's knowledge never been deployed in the method as presently claimed. For example, in Johnson, C. A., “Applications of Computational Homology,” Marshall Digital Scholar, Marshall University, Jan. 1, 2006, the author reviews manipulation of complex homological surfaces such as can, without limitation, be generated with the present invention. In compiling the present data capture when the data compilation will naturally create a homological surface representing multiple dimensions (data), an n-dimensional simplicial complex K constitutes a collection of simplices of dimension≤n, where n is 100, such that:

If l is a face of a simplex k [symbol] K, then l [symbol] K.
For k, l∈K, k∩l [symbol] k [symbol] k∩l [symbol] l.

However, when homological surfaces do not need to be taken into account, such as for mapping of multiple variables over time as, for example, musical aggregations, the following equation is used to implement the automated computation of data compiling and mapping:

x=Σ 7+y (v_{n1, n2, n3, n4, n5, n6, n7 . . .} ), where x is the output resulting from the compiled and mapped data capture (solved for by the equation);
y=0-100; and
n=patient (or animal) vital sign or indicator with n1, n2, n3 . . . representing individual vital signs or indicator variables including, optionally, time.

In the context of the previous equation, “Σ” means either summation or aggregation.

When implementing automated data compiling and mapping with either, or both, of the above equations, the patient (vital) signs or indicators, compiled and mapped, may be between 7 and 100. While the compiling and mapping is automated, the selection of 7-100 signs or indicators is discretionary on the part of the physician or health care provider—and the invention operates—to render gestalt data—regardless of which signs or indicators are selected.

The present invention, thus allowing an accumulated, compiled and tensorialized “Manifold Medical Data-Set” to be displayed mathematically, visually, sonically or musically, enjoys utility and is medically valuable because it allows for mathematical, visual, sonic, or musical recognition of patterns otherwise undiscerned. The present invention, therefore, is useful to a medical diagnostician in developing a therapeutic analysis leading to highly individualized treatment protocol(s). In addition, the present invention provides a novel and heretofore unknown method of programmatic and axiomatic musical composition in the style of “Tone Rows” of musical composers.”

Further, the present invention has applicability not only to pattern recognition for data sets, generally of multiple human or animal subjects, but also in the pattern depiction of data-over-time for an individual subject. For example, in the Exemplification shown below, four months of four patient data parameters, stipulated as to date and time, are listed in a table array. For a health care provider, who is used to considering static assessments of blood pressure, oxygenation, weight and so forth, historically it has been difficult to view a time-based array of such data to see where the meaningful fluctuations are. In the case of an individual's signs and symptoms—those skilled in the art already know what patterns to look for over time—how can those signs and symptoms be depicted for easy recognition when a page full of raw data are so hard to read, especially to read quickly and accurately?

The answer is that an enhanced graph or musical depiction, according to the present invention, makes the data progression pattern quickly discernible to the eye or ear. Consider, for example, as shown in the Exemplification, that there is a cyclical nature to the variance in blood pressure systolic/diastolic values. It certainly is possible for a health care provider to “run the eye” down the column of values to discern the fluctuation and its significance. It is very much easier, and less likely to produce error, for the health care provider to see the pattern in the data in an enhanced graph, or to hear it as sound/music, to discern the data patterns that are less easy to see in a data array or matrix.

EXEMPLIFICATION: PATIENT RIOCO J'HANCIOCIOLA
(TAMPA BAY, FLORDIA) Sporadic & Select Vitals 2019

| Date | Time | BPsd | % SpO₂ | PRbPm | ΣW |
|---|---|---|---|---|---|
| . . . January | | | | | |
| 27 | 130a | 70/40 | 97 | 39 | 205 |
| 27 | 1130a | 70/50 | 93 | 60 | 208 |
| . . . February | | | | | |
| 20 | 530a | 63/47 | 98 | 55 | 205 |
| 20 | Noon | 100/58 | 98 | 49 | 204 |
| 21 | 6a | 60/50 | 98 | 45 | 204 |
| 22 | 2p | 100/70 | 99 | 60 | 204 |
| . . . March | | | | | |
| 02 | 4a | 120/70 | 98 | 82 | 204 |
| 03 | 3a | 60/48 | ? | 77 | 203 |
| 03 | 9a | 70/50 | 97 | 75 | 203 |
| 03 | Noon | 80/55 | 98 | 60 | 202 |
| 04 | 10a | 90/55 | 98 | 60 | 203 |
| 04 | 2p | 70/55 | 98 | 57 | 203 |
| 06 | 9a | 80/50 | 99 | 70 | 202 |
| 07 | 4p | 75/50 | 97 | 67 | 206 |
| 08 | 6a | 75/55 | 99 | 65 | 204 |
| 10 | 11a | 75/50 | ? | 62 | 207 |
| 10 | 430p | 80/55 | 98 | 85 | 206 |
| 10 | 7p | 85/50 | 97 | 46 | 205 |
| 10 | 10p | 75/40 | 97 | 48 | 204 |
| 14 | 3p | 70/50 | 98 | 58 | 206 |
| 15 | 7a | 75/50 | 97 | 70 | 203 |
| 17 | 1a | 75/45 | 98 | 48 | 202 |
| 20 | 3a | 85/60 | 97 | 60 | 207 |
| 20 | 11a | 80/60 | 97 | 55 | 205 |
| 21 | 3p | 100/60 | 98 | 86 | 208 |
| 24 | 7a | 99/64 | 97 | 68 | 208 |
| 24 | 930p | 95/60 | 95 | 44 | 205 |
| 25-01 | Unable to Track . . . | | | | |
| . . . April | | | | | |
| 02 | 2a | 65/55 | ? | ? | 207 |
| 03 | 5a | 53/38 | 97 | 65 | 206 |
| 09 | Noon | 99/64 | 97 | 55 | 211 |
| 10 | 10p | 65/50 | 97 | 70 | ?? |
| 15 | 2p | 120/70 | 99 | 62 | 209 |
| 16 | 530a | 70/50 | 97 | 50 | 209 |
| 16 | 9p | 100/50 | 98 | 50 | 208 |
| 17 | 2p | 65/40 | 97 | 70 | 208 |
| 18 | 1030a | 125/80 | 98 | 50 | 208 |
| 19 | 1030a | 58/43 | 92 | 70 | 207 |
| 20 | 3p | 70/45 | 97 | 45 | 207 |
| 21 | 230p | 56/36 | 97 | 65 | 206 |
| 22 | 5p | 68/40 | 97 | 52 | 205 |
| 30 | 3p | 120/80 | 98 | 70 | 202 |
| . . . May | | | | | |
| 04 | 1p | 100/70 | 98 | 70 | 208 |
| 05 | 12a | 95/65 | 98 | 70 | 207 |
| 06 | 1p | 85/55 | 97 | 55 | 208 |
| 07 | 1p | 100/60 | 98 | 60 | 208 |
| 10 | 11a | 120/85 | 93 | 89 | 208 |
| 11 | 11a | 100/70 | 97 | 80 | 207 |
| 12 | 1a | 100/65 | 98 | 100 | 206 |
| 13 | 5a | 110/75 | 97 | 80 | 208 |
| 16 | 11a | 90/55 | 97 | 45 | 210 |
| 18 | 9p | 75/50 | 98 | 52 | 206 |
| 19 | 130a | 90/44 | 97 | 50 | 205 |
| 21 | 5p | 65/40 | 99 | 85 | 203 |
| 30 | 9p | 70/40 | 97 | 55 | 205 |

The value of the enhanced graph or audible patterns does not stop with the diagnostician. A patient can benefit from hearing his or her own data renditions—or seeing the enhanced graphing of them—by recognizing certain patterns which then may be a representation of signs or symptoms the individual wishes to change. Considering again the blood pressure example, if a goal is to even out fluctuations in blood pressure, at successive health consultations the individual can listen to the current blood pressure trends or look at them with enhanced graphics. If interventions to smooth out blood pressure variation have been successful, the patient will later see or hear the new pattern and enjoy the positive feedback. Also, no one likes to be nagged, and it is quite different to hear someone cajole “Lose weight! Stop smoking!” (negative), then it is to see or hear the pleasant visual or tonal adjustments of, say, weight reduction, bad cholesterol improvement, and so forth (positive). Alongside this wellness and biofeedback benefit, the graphic or musical rendition for each patient is an independent “fingerprint” of patient data each time, and so is unique to the patient.

Thus, the present invention provides a graphic or music/sound way for a person not only to hear a depiction of their unique identity, but to have an entirely new way “of looking in a mirror,” for self-assessment or for registry of individuality by others.

Pattern analogizing between individual patients and patient populations, or among patient populations, is still in development at this writing. The significance of certain patterns in data as to populations will be developed with this inventive technique, which is not an abstraction and does not use any conventional steps. In other words, the present invention as to populations is not a pie-in-the-sky aspiration of finding patterns in clinical study data sets. Instead, the present invention is a way of organizing and displaying data—data already understood to contain its own multiple meanings that can be assessed by means already known in the art—in a way that can be more easily characterized, categorized, rendered and archived than data presentation means of the prior art.

Without question, the present invention improves the efficiency of computers tasked with rendering outputs of data sets, due to the simplification of the data into enhanced graphics or music/sound in ways which reduce computation needed and therefore speeds up processing. Moreover, the ability to depict individual patient data over time already exists, and can literally be generated by, as a nonlimiting example, inputting the data from the Exemplification provided herein into a music program such as GuitarPro, to generate a music/sound file after assigning a music parameter to each data type—or analogously by assigning each data type to a parameter in an enhanced graphing protocol for visual output.

Overarchingly, therefore, the present invention addresses two separate universes of trends: (1) the dynamic model of single patient data (multiple data types) over a specified time period, for pattern depiction; and (2) the clinical study model of identifying patterns in populations by assessing (multiple) patient data over time the same way. With the individual, there may or may not be an existing diagnosis to which data patterns can be mapped. In populations, there likely will already be a common diagnosis which can, in turn, be mapped to patterns that emerge from the patient population data—in enhanced graphing or in music/sound trends.

While the invention has been particularly shown and described with reference to embodiments described above, it will be understood by those skilled in the art that various alterations in form and detail may be made therein without departing from the spirit and scope of the invention, as defined by the appended claims.

What is claimed is:

1. A method for medical diagnosis comprising the steps of:
electing at least seven vital signs or indicators of an animal or human for which diagnosis is indicated;
monitoring said vital signs or indicators over time with data capture; and
compiling and mapping said data, using a computer, to map said data capture in a form selected from the group consisting of a mathematical tensor, a visual graph and a musical composition.

2. The method according to claim 1, wherein said form further comprises (1) a linear graph of x, y, z, as x=t\hat{ }y=P/p; x=t\hat{ }y=pBpm\hat{ }z=dP; (2) a hyperbolic graph; (3) a symbolic logic graph; or (4) a logarithmic graph.

3. The method according to claim 1, wherein said compiling and mapping are calculated according to the equation:
x=Σ 7+y (v_{n1, n2, n3, n4, 5, n6, n7 . . .} ), wherein x is the output to a user;
y=0-100; and
n=individual vital sign or indicator data or time.

4. The method according to claim 1, wherein said compiling and mapping are calculated according to homologic equations:
If l is a face of a simplex k [symbol] K, then l [symbol] K.
For k, l∈K, k∩l [symbol] k [symbol] k∩l [symbol] l.

5. The method according to claim 3, wherein n is selected from the group consisting of systolic pressure, diastolic pressure, body temperature, time elapse, oximeter, pulse, body mass, antibody titer, white cell count, T cell count, red cell count, a cancer marker, hematocrit, hemoglobin, platelet count, platelet volume, an immune marker, a metabolic marker, and an indicator.

6. The method according to claim 5, wherein said indicator is a marker.

7. The method according to claim 1, wherein said at least seven vital signs include (1) systolic pressure values of 60-140; (2) diastolic pressure values of 40-85; (3) time of day values of 1-24; (4) oximeter data values of 92-99; (5) date data values translated to adjusted circadian process; (6) pulse data values of 40-90; and (7) weight values.
